(19) United States
(12) Patent Application Publication
(10) Pub. No.: US 2021/0235911 A1
(43) Pub. Date: Aug. 5, 2021

(54) DEVICES AND METHODS FOR APPLYING COMPRESSION SOCKS TO FEET

(71) Applicant: Paul G. Sommer, Valparaiso, IN (US)

(72) Inventor: Paul G. Sommer, Valparaiso, IN (US)

(21) Appl. No.: 17/167,691

(22) Filed: Feb. 4, 2021

Related U.S. Application Data
(60) Provisional application No. 62/969,959, filed on Feb. 4, 2020.
(60) Provisional application No. 63/017,723, filed on Apr. 30, 2020.
(60) Provisional application No. 63/106,709, filed on Oct. 28, 2020.

(51) Int. Cl. A47G 25/90; A61F 13/08

(52) U.S. Cl. CPC A47G 25/905; A41B 2400/44; A61F 13/08

(57) ABSTRACT

Devices and methods for assisting a user to don and remove a compression sock from the user's leg. The device includes a base, at least two side members secured to the base on opposite sides thereof, and multiple arms extending from the base in a direction along the longitudinal axis and between the side members. Each of the arms has a midpoint that curves inward toward the longitudinal axis of the device, and terminates at a distal end. The arms in combination form a composite shape that has a midportion that curves inward toward a longitudinal axis of the device to define a neck, defines an interior that is surrounded by the midportions of the arms and within which the longitudinal axis of the device is disposed, and defines an opening surrounded by the distal ends of the arms.

### CROSS REFERENCE TO RELATED APPLICATIONS

This application claims the benefit of U.S. Provisional Application Nos. 62/969,959 filed Feb. 4, 2020, 63/017,723 filed Apr. 30, 2020, and 63/106,709 filed Oct. 28, 2020. The contents of these patent documents are incorporated herein by reference.

### BACKGROUND OF THE INVENTION

The present invention generally relates to compression socks, stockings, sleeves, etc. (collectively, referred to herein as socks) and their use. The invention particularly relates to systems and methods suitable for aiding users in applying medical grade high pressure or low pressure compression socks to their feet and legs.

Lower extremity conditions such as leg edema, venous insufficiency, varicose veins, cellulitis, and deep venous thrombosis affect millions of people and cost billions of dollars in health care expenditures and insurance costs. Currently, the leading cause of insufficient treatment of these conditions is the lack of compliance in patients' use of prescribed compression socks (also referred to as compression stockings or hoses) due at least in part to the manual difficulty in putting on the socks, into which an individual must typically force their entire lower leg against the resistance of the compression forces generated by the sock. Often, patients simply do not have the finger, hand, or arm strength and/or lack sufficient flexibility and coordination with which to put on compression socks. Due to this lack of compliance, people may retain fluid edema within their legs, leading to excessive pressure against the skin, dermal capillaries, and subcutaneous circulation potentially leading to a breakdown of skin and subcutaneous tissue. This may promote venous leg ulcerations and the stasis (pooling) of fluid which leads to bacterial reproduction and infection, for example, cellulitis. Due to noncompliant use of compression socks, more and more vascular surgeons are opting for vein ligation despite the inherent risks, complications, and health care costs associated with this procedure.

Various devices are available to assist individuals with the use (specifically, the application or donning) of compression socks. However, these devices are often difficult to coordinate and use. For example, a common type of device includes a lightweight and flimsy combination of a plastic sleeve and a rope to pull up the sock. This device requires tremendous hand strength and the coordinated use of two hands to both handle the device and stretch the sock over it. Very often the types of individuals who need compression socks, such as the elderly, often do not have the hand and grip strength required to pull this rope or the coordination of two hands in which to do this. During use, these devices are also prone to slipping out from under the sock and therefore need to be reapplied.

Another approach to easing the application of a compression sock is to equip the sock with a side zipper so that only the feet must be forced into the sock, after which the sock can be zipped with the side zipper. This approach is not entirely effective because adequate material tension required of a compression material results in the zipper being difficult to operate, especially since the user can only use two fingers to grasp and pull the zipper, and the user is often not strong enough to pull the zipper against the compressive strength of the material. The zipper itself is unattractive, which can further contribute to noncompliance of use.

Yet another approach to easing the application of a compression sock is to begin with a flat piece of material with interlacing flaps that are equipped with hook-and-loop fasteners (e.g., VELCRO®) and alternate in direction, such that the leg can be wrapped with the compression sock by slowly interlocking and tightening the flaps. Significant problems with this approach are that users may not be able to bind the sock to apply adequate pressure, and the pressure may not be evenly dispersed relative to what can be achieved with a conventional compression sock. If the compression tension is tighter proximally than it is distally, the compression may create an adverse tourniquet effect.

Other less frequently used techniques include the use of dishwashing rubber gloves in an attempt to have a better grip on a compression sock. This does not eliminate the need for finger strength, coordination, and manipulation (dexterity) to apply the sock, and can complicate the procedure due to there being less tactile sense due to the rubber gloves.

Patients may also resort to applying a compression sock that has been turned inside-out except for the foot portion including the heel. This approach also does not eliminate the need for finger strength, coordination, and manipulation, and has the added drawback of significantly increasing the tightness of the foot portion of the sock, which is often the most difficult part of the sock to put on when using the fingers.

In view of the above, it can be appreciated that there are certain problems, shortcomings or disadvantages associated with the prior art, and that it would be desirable if devices and methods were available for applying compressions socks that were easier to use than previously available.

### BRIEF DESCRIPTION OF THE INVENTION

The present invention provides devices and methods suitable for assisting a user in applying a compression sock to their foot.

According to one aspect of the invention, a device is provided that includes a base, a longitudinal axis sufficiently coinciding with a center of the base so that the device is stable when the device is supported by the base on a surface, at least two side members secured to the base on opposite sides thereof, and multiple arms extending from the base in a direction along the longitudinal axis and between the side members. Each of the side members has a handle and extends from the base along the longitudinal axis. Each of the arms has a midpoint that curves inward toward the longitudinal axis of the device, and terminates at a distal end. The arms in combination form a composite shape that has a midportion that curves inward toward the longitudinal axis of the device to define a neck, defines an interior that is surrounded by the midportions of the arms and within which the longitudinal axis of the device is disposed, and defines an opening surrounded by the distal ends of the arms.

According to another aspect of the invention, a method is provided for covering at least a portion of a user's leg with a compression sock that has a foot portion including a toe portion, has a leg portion extending from the foot portion, and has an upper end that defines an opening into an interior of the compression sock. The method includes providing a device that includes a base, a longitudinal axis sufficiently coinciding with a center of the base so that the device is stable when the device is supported by the base on a surface, at least two side members secured to the base on opposite sides thereof, and multiple arms extending from the base in a direction along the longitudinal axis and between the side members. Each of the side members has a handle and extends from the base along the longitudinal axis. Each of the arms has a midpoint that curves inward toward the longitudinal axis of the device, and terminates at a distal end. The arms in combination form a composite shape that has a midportion that curves inward toward the longitudinal axis of the device to define a neck, defines an interior that is surrounded by the midportions of the arms and within which the longitudinal axis of the device is disposed, and defines an opening surrounded by the distal ends of the arms. Thereafter, the method includes placing at least a portion of the compression sock within the interior of the composite shape, expanding the opening of the compression sock radially outward and over the distal ends of the arms, and pushing the upper end of the compression sock over the arms toward the base of the device such that at least the upper portion of the composite shape defined by the arms is surrounded by the compression sock and the foot portion of the compression sock is adjacent the opening of the composite shape. Thereafter, the method includes inserting the user's foot, toes first, into the foot portion of the compression sock at the opening of the composite shape and moving the foot through the interior of the composite shape, along the longitudinal axis of the device, and toward the base of the device. As the user inserts the user's foot through the interior of the composite shape toward the base of the device, the compression sock is applied to the user's foot and then the user's leg as the compression sock slides over the arms and the distal ends thereof until the compression sock and the upper end thereof entirely slips over the distal ends of the arms and onto the user's leg.

Technical effects of the device and method described above preferably include the ability to apply a compression sock to a user's foot with a reduced requirement for physical strength relative to previous devices and methods. Preferably, the device is constructed to have sufficient strength and stability to greatly reduce the need for finger and hand strength of the user and reduce the need for whole body flexibility and coordination when applying a compression sock.

Other aspects and advantages of this invention will be appreciated from the following detailed description.

### BRIEF DESCRIPTION OF THE DRAWINGS

FIGS. 1 and 2 represent perspective and side views of a device configured for assisting a user in applying a compression sock to the user's leg in accordance with certain nonlimiting aspects of a first embodiment of the invention.

FIGS. 3 and 4 represent perspective and side views of a device configured for assisting a user in applying a compression sock to the user's leg in accordance with certain nonlimiting aspects of a second embodiment of the invention.

FIG. 5 represents a perspective view of a device configured for assisting a user in applying a compression sock to the user's leg in accordance with certain nonlimiting aspects of a third embodiment of the invention.

FIGS. 6 and 7 represent various views of removal clips that are configured for use in combination with the device of FIG. 5 to assist in removing a compression sock from the user's leg in accordance with certain nonlimiting aspects of the invention.

FIGS. 8 through 11 represent various aspects of a first embodiment of a loading device that may be used with the devices of FIGS. 1 through 5 in accordance with certain nonlimiting aspects of the invention.

FIGS. 12 through 16 represent various aspects of a second embodiment of a loading device that may be used with the devices of FIGS. 1 through 5 in accordance with certain nonlimiting aspects of the invention.

### DETAILED DESCRIPTION OF THE INVENTION

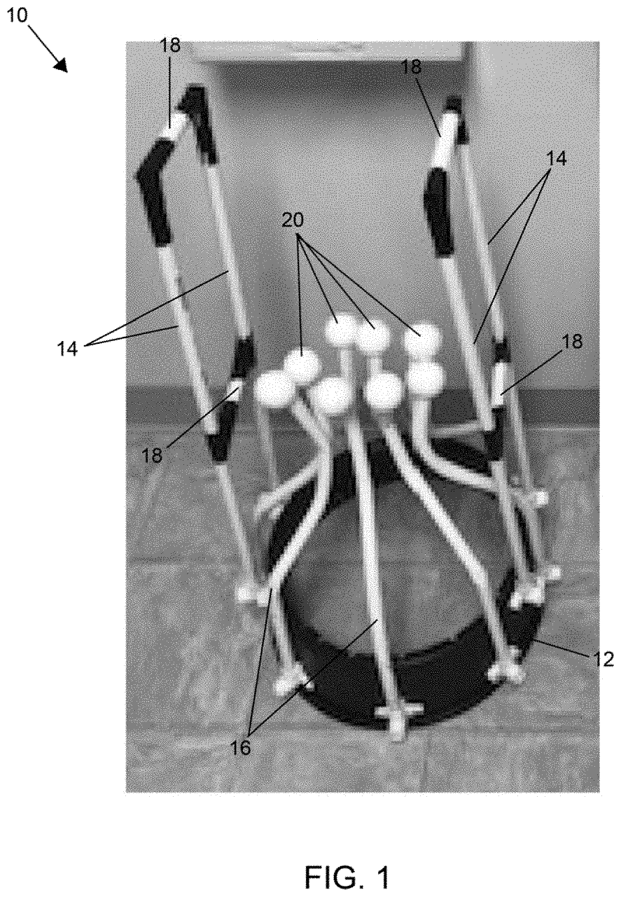

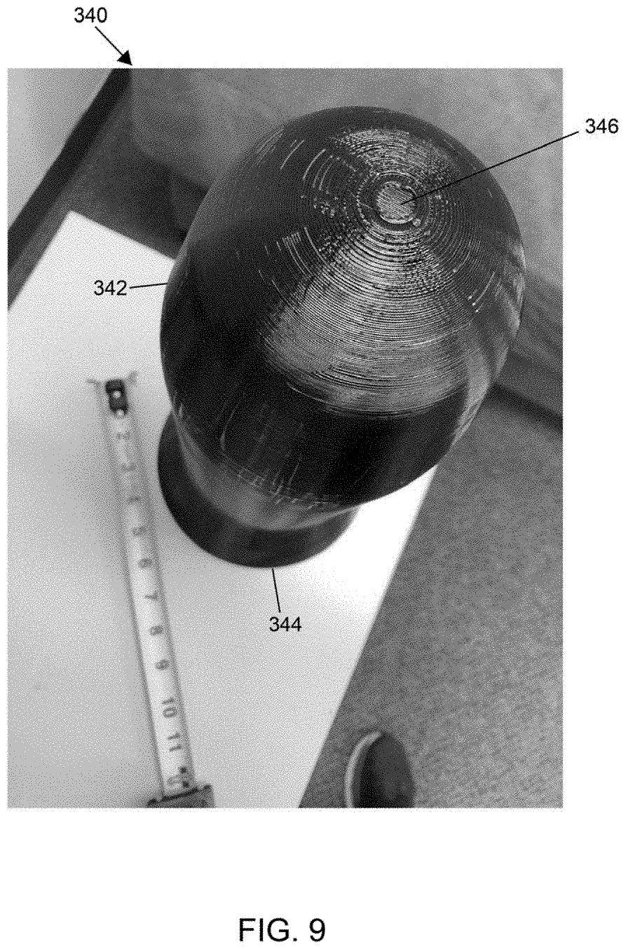

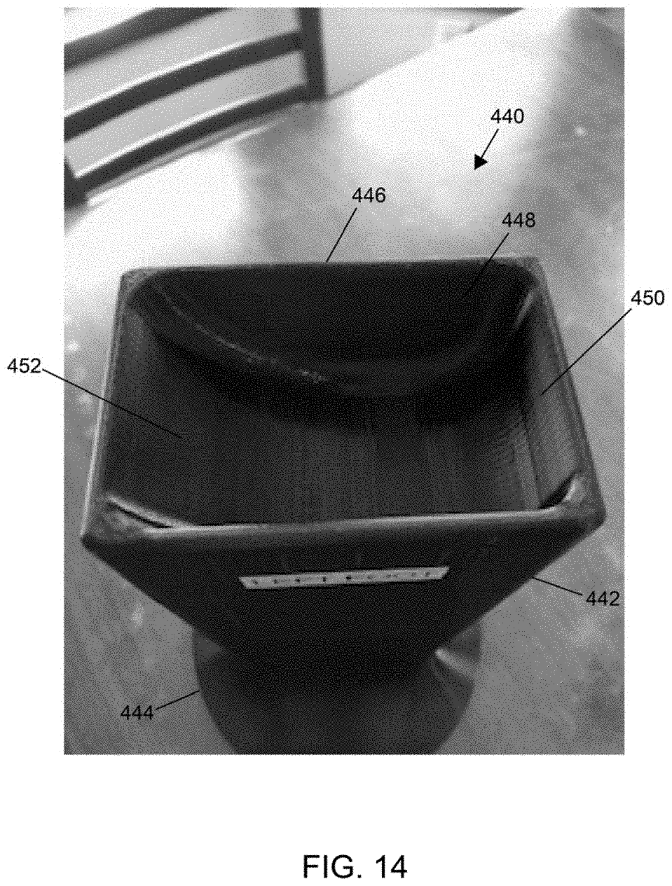

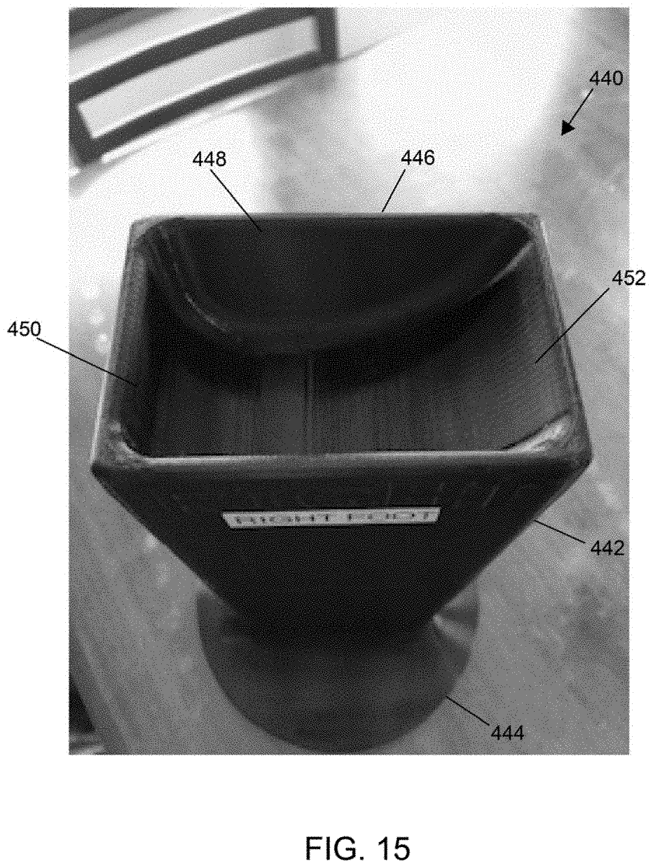

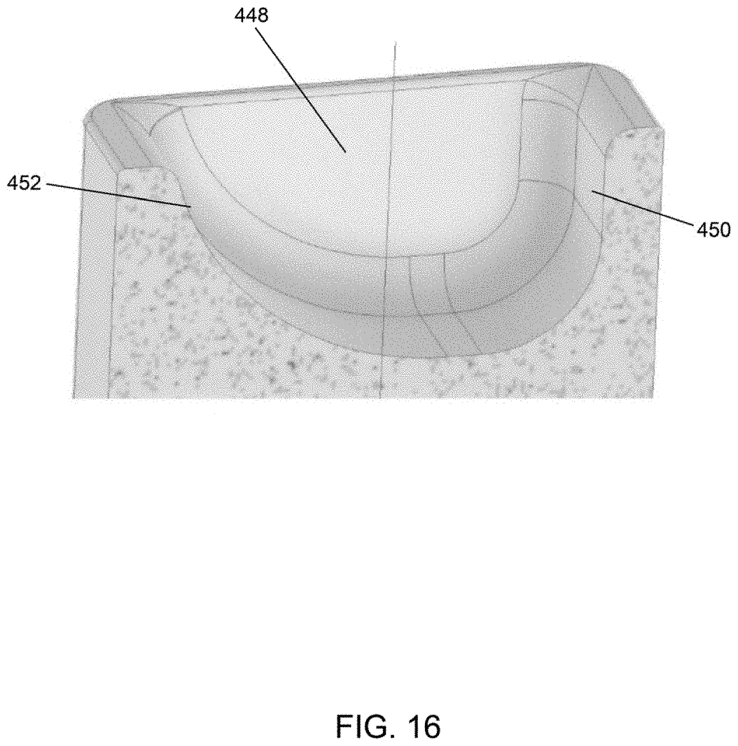

FIGS. 1 through 16 represent aspects of nonlimiting devices configured for assisting a user in putting on a compression sock onto the user's foot and/or lower leg. To facilitate the description provided below of the embodiment represented in the drawings, relative terms, including but not limited to, “vertical,” “horizontal,” “lateral,” “front,” “rear,” “side,” “top,” “bottom,” “upper,” “lower,” “above,” “below,” “right,” “left,” etc., may be used in reference to the orientation of the devices as represented in FIGS. 1-5, 10, and 11, and therefore are relative terms that are useful to describe the structure and operation the devices represented in the drawings, but should not be necessarily interpreted as limiting the scope of the invention. It should also be noted that the drawings are drawn for purposes of clarity when viewed in combination with the following description, and therefore are not necessarily to scale.

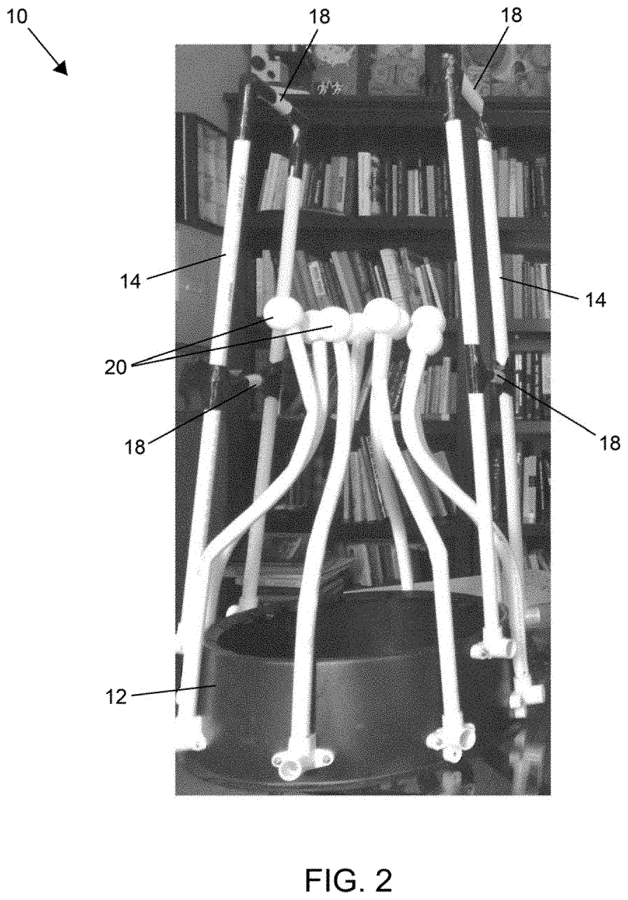

The device 10 of FIGS. 1 and 2 includes a rigid base 12 at a lower end, a pair of rigid side members 14 coupled to the base 12 that extend upward from opposite sides of the base 12, and a plurality of flexible or semi-rigid arms (struts) 16 coupled to the base 12 and extend upward therefrom toward an upper end of the device 10. The base 12 is represented as annular or ring-shaped to define a central opening 22, though other shapes having a central opening are foreseeable. The side members 14 may comprise a rectangular frame to define one or more handles 18, preferably parallel to a plane containing the base 12. The side members 14 may be adjustable to allow selective modification of the distance of the handles 18 from the base 12. Additionally, the side members 14 may be removable or configured to telescopically collapse toward the base 12.

The arms 16 are preferably equally spaced about the base 12 and define a shape about a longitudinal axis 30 of the device 10 that coincides with or is near the center of the base 12. The arms 16 may define various shapes configured to receive and hold a compression sock thereon. Preferably, the arms 16 generally define a partial hourglass or bullet nose shape. In the embodiments shown, each arm 16 has a midportion that curves inward toward the longitudinal axis 30 of the device 10, has an upper portion that curves outward away from the longitudinal axis 30, and terminates at a distal end 20 thereof. In combination, the arms 16 form a composite shape that has a bulbous midportion that curves inward toward the longitudinal axis 30 of the device 10 to define a neck, has an upper portion that flares outward away from the longitudinal axis 30, defines an interior that is surrounded by the midportions and upper portions of the arms 30 and within which the longitudinal axis 30 of the device 10 is disposed, and defines an opening surrounded by the distal ends 20 of the arms 16. The composite shape is represented as having a circular cross-section, though a circular shape is not required. Each of the arms 16 is represented as terminating with a spherical ball at its distal end 20. The device 10 comprises at least two arms 16, more preferably four to eight arms 16, although other numbers of arms 16 are possible.

During use, the device 10 may be placed such that the base 12 rests on a surface capable of supporting the device 10, such as the ground, floor, or a table. Preferably, the surface is flat such that the base 12 is stable. A compression sock is installed on the arms 16 such that the sock is inverted and inside-out and so that the sock surrounds, extends over, and partially encloses at least the upper portions of the arms 16. The tapered neck defined by the arms 16 is configured such that there is a relatively small area over which the sock must be initially stretched and gathered over, preferably with the toe of the sock being slightly indented at the opening of the composite shape into which the toe is initially received.

A method of using the device 10 will be described in reference to a conventional compression sock that has a foot portion including a toe portion, has a leg portion extending from the foot portion, and has an upper end that defines an opening into an interior of the compression sock. Nonlimiting examples of compression socks include medical grade high-pressure or low-pressure compression socks. The sock is installed on the device 10 by placing at least a portion of the compression sock within the interior of the composite shape formed by the arms 16, expanding the opening of the compression sock radially outward and over the distal ends 20 of the arms 16, and pushing the upper end of the compression sock over the arms 16 toward the base 12 of the device 10 such that at least the upper portion of the composite shape defined by the arms 16 is surrounded by the compression sock and the foot portion of the compression sock is adjacent the opening of the composite shape. Thereafter, the user inserts their foot, toes first, into the foot portion of the compression sock at the opening of the composite shape, and then moves their foot through the interior of the composite shape, along the longitudinal axis 30 of the device 10, and toward the base 12 of the device 10. As the user inserts their foot through the interior of the composite shape toward the base 12 of the device 10, the compression sock is applied to the foot and then the user's leg as the compression sock slides over the arms 16 and their distal ends 20 until the compression sock and the upper end thereof have entirely slipped over the distal ends 20 and onto the user's leg. Once the sock is entirely donned on the user's foot and leg, the user can remove their leg from the device 10 through the opening at the upper end of the composite shape by raising their leg and/or pushing down or away with the handles 18.

Preferably, the base 12 is sufficiently stable on a surface such that the user may use both hands when slipping the sock over the arms 16. Once the inverted sock has been slipped over the top of the arms 16, it is preferably retained thereon due in part to the shape of the arms 16 and the spherical balls at the distal ends 20 thereof. The user may grip the handles 18 on the side members 14 to further stabilize the device 10 and/or pull the device 10 upwards to assist in inserting their leg into the device 10. As the user inserts their foot into the device 10, the sock will slide off the arms 16 and simultaneously cover their foot. Preferably, the distal ends 20 of the arms 16, including the spherical balls, are formed of a relatively low friction material to promote sliding of the sock.

Use of the handles 18 throughout the process allows the user to use the strength of their arms and hands for applying the sock to their foot rather than relying solely on, for example, the strength of their fingers, thereby promoting the ease of application and coordination. The device 10 may be used while the user is sitting or standing. The device 10 is durable, simple to manufacture, and easy to use.

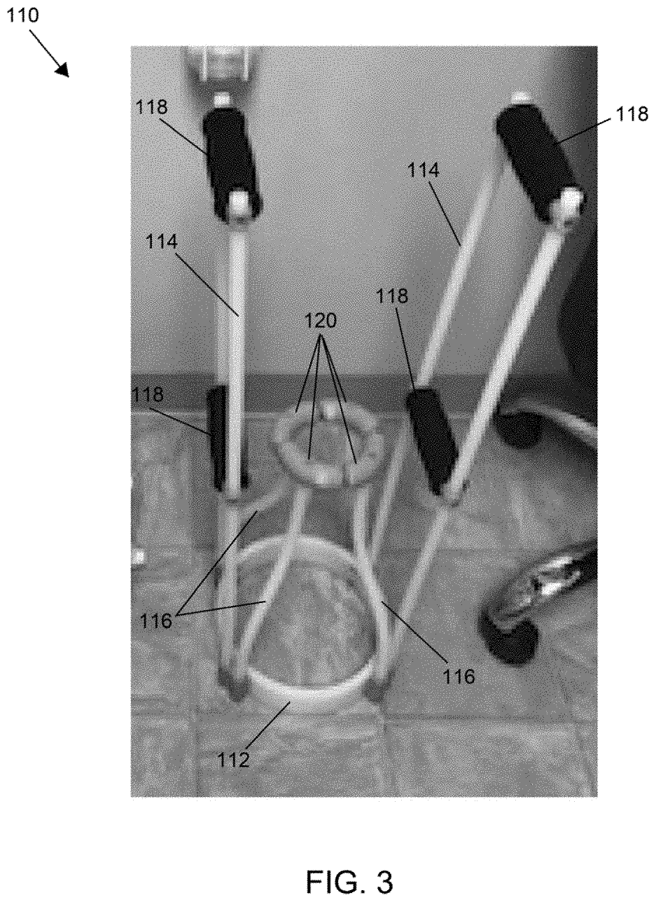

FIGS. 3 through 5, 10 and 11 depict additional configurations of the device 10 of FIGS. 1 and 2 in accordance with further embodiments of this invention. In view of similarities between the embodiment of FIGS. 1 and 2 and the embodiments of FIGS. 3 through 5, 10, and 11, the following discussion of FIGS. 3 through 5, 10, and 11 will focus primarily on aspects of the further embodiments that differ from the first embodiment in some notable or significant manner. Other aspects of the further embodiments not discussed in any detail can be, in terms of structure, function, materials, etc., essentially as was described for the first embodiment. In FIGS. 3 through 5, 10, and 11, consistent reference numbers are used to identify components that are the same or functionally related to components identified in FIGS. 1 and 2, but with a numerical prefix (e.g., 1, 2, etc.) added to distinguish components depicted in FIGS. 3 through 5, 10, and 11 from their counterparts depicted in FIGS. 1 and 2.

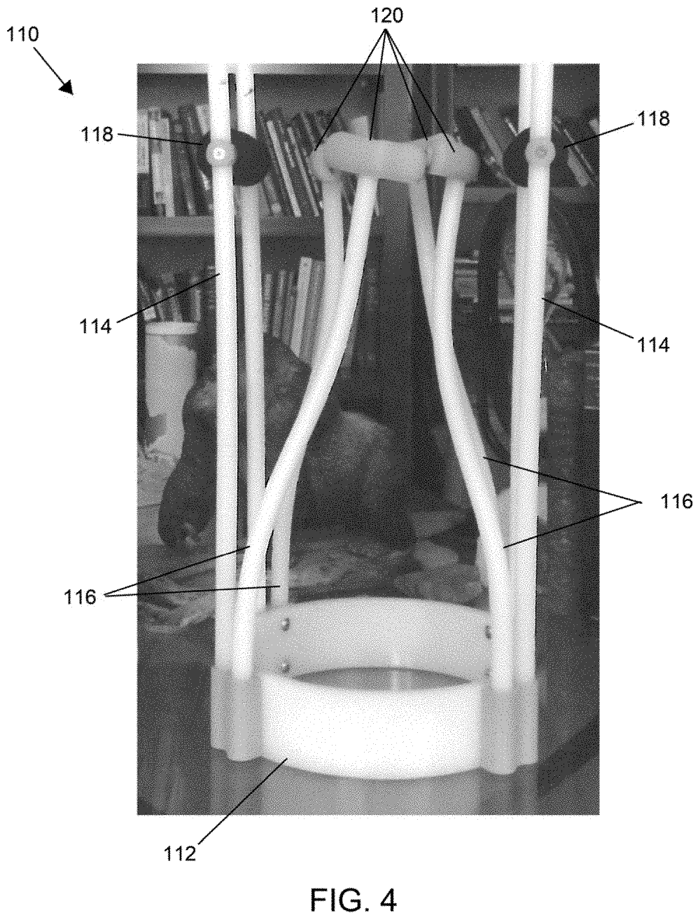

FIGS. 3 and 4 depict a second embodiment of a device 110 which replaces the spherical distal ends 20 of FIGS. 1 and 2 with soft flexible serrated members 120 that have an arcuate shape so that, together, the soft flexible members 120 define a generally circular opening at the entrance to the arms 116. Each of the soft flexible members 120 are mounted to an individual arm 116. Under certain circumstances, the soft flexible members 120 at the distal ends of the arms 116 may promote the ease with which a compression stocking can be installed on the device 110.

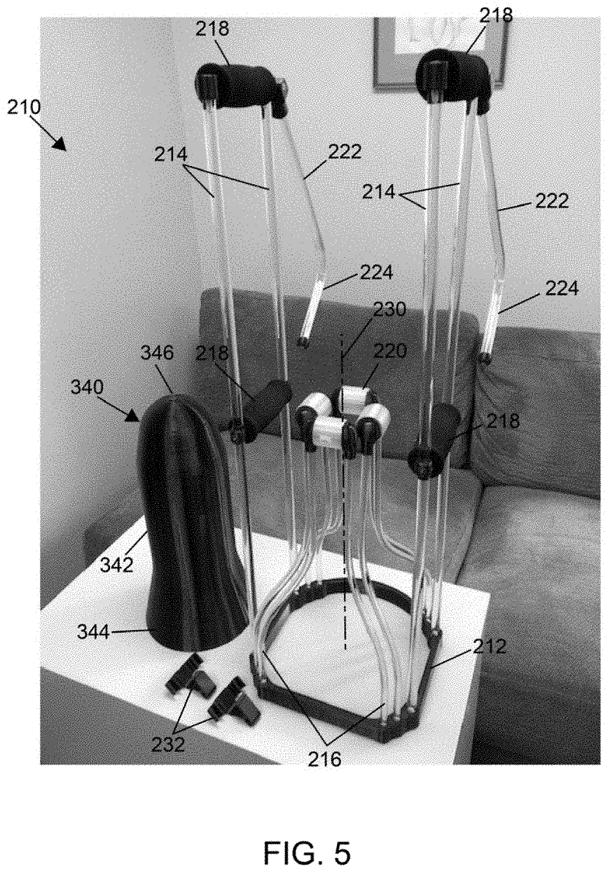

FIGS. 5, 10, and 11 depict a third embodiment of a device 210 which replaces the spherical distal ends 20 of FIGS. 1 and 2 and the soft flexible serrated members 120 of FIGS. 3 and 4 with rollers 220. Each roller 220 is connected to the distal ends of two adjacent arms 216 such that the device 210 comprises eight arms 216 and four rollers 220 that define a generally square-shaped opening at the distal ends of the arms 216 rather than the circular-shaped opening of the embodiment of FIGS. 1 through 4. The axial ends of each roller 220 may be rounded to eliminate sharp corners. The rollers 220 are preferably capable of freely rotating to further decrease the active friction between the distal ends of the arms 216 and the compression sock as it is being applied. The two arm connections to each of the rollers 220 can also provide greater stability during use while also permitting a desirable degree of lateral or outward flexible motion of the arms 216 in order for the arms 216 to expand and allow passage of the leg fitting through the opening within and surrounded by the arms 216.

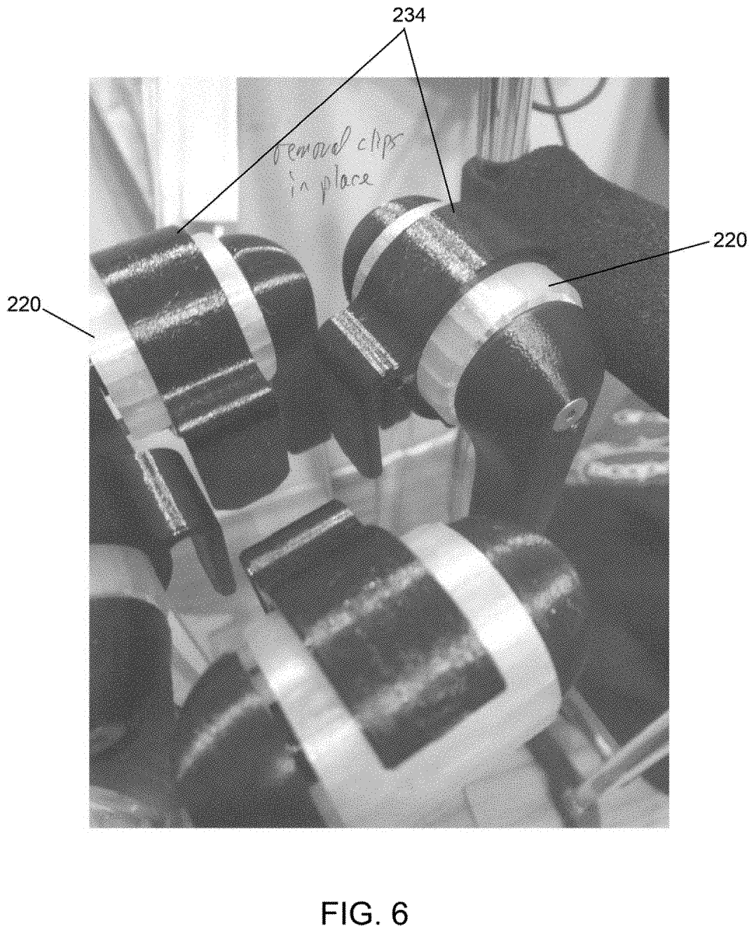

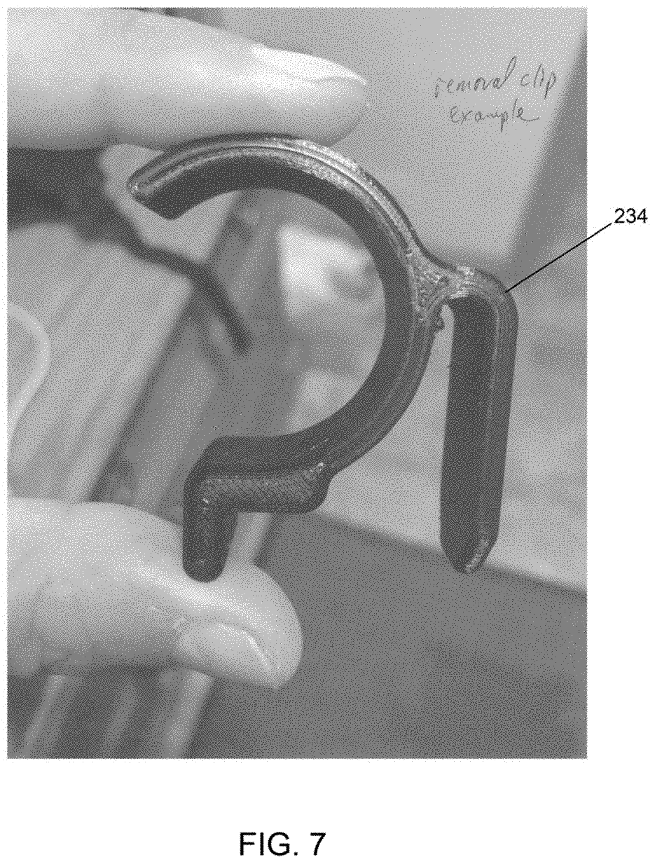

FIGS. 6 and 7 represent various aspects of nonlimiting removal clips 334 that may be used with the rollers 220 of the device 210. These removal clips 334 are configured to be attached to the rollers 220 and simultaneously fasten or otherwise grip a sock around its opening while the sock is worn by a user to facilitate the removal of the sock from the user's leg as the user pushes the handles 218 of the device 210. In particular, the user inserts their leg donning a sock into the opening of the device 210 within and surrounded by the arms 216, after which the clips 334 are used to secure the proximal edge of the sock to each of the rollers 220. The user is then able to push on the handles 218 so that, as the sock remains secured to the rollers 220 with the clips 334, the sock can be gradually removed from the user's leg and foot.

While the devices 10, 110, and 210 may be used while the user is sitting or standing, it is generally preferred for the user to be seated while inserting their foot into a compression sock for improved stability. Further, there may be benefits to using the devices 10, 110, and 210 while seated. For example, as the user inserts their foot into the device 10, 110, or 210, the user may pull the device 10, 110, or 210 toward themselves at an inclined angle relative to the surface on which it rested. Such action promotes ease of removal of a loading device (described hereinafter) being extruded or pushed out of the bottom of the device 10, 110, or 210 through the opening in the base 16, 116, or 216.

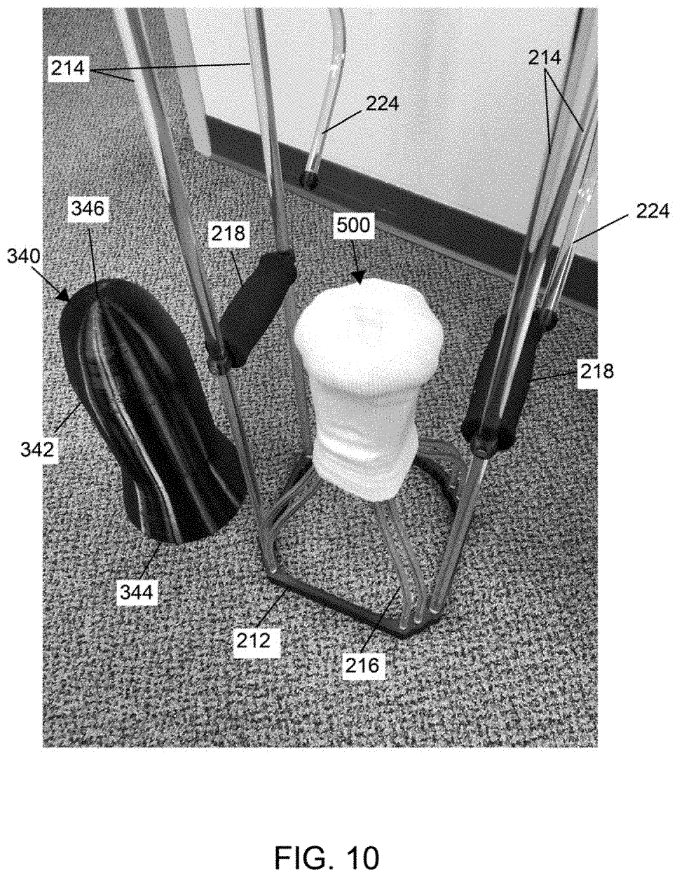

The devices 10, 110, and 210 may include additional features configured to promote ease of use while sitting. For example, FIGS. 5 and 10 represent the device 210 as including a pair of extended shafts 222 each having a handle 224 that are coupled to each of the side members 214. Optionally, the shafts 222 may be coupled to the side members 214 with pivoting joints. If the user intends to apply a compression sock from a seated position, the handles 224 may be used to extend the user's reach while attempting to tilt the device 210 toward the user.

The devices 10, 110, and 210 may further include holders configured to releasably secure a compression sock to the arms 16, 116, or 216. For example, FIG. 5 represents holders 232 that are configured to releasably couple to one or more of the arms 216 of the device 210 and include a clip or other fastener capable of releasably coupling to a compression sock. Optionally, the holders may be configured to slidably couple to the arms 16, 116, or 216 such that they may freely slide along the arms 16, 116, or 216 while holding a compression sock thereto.

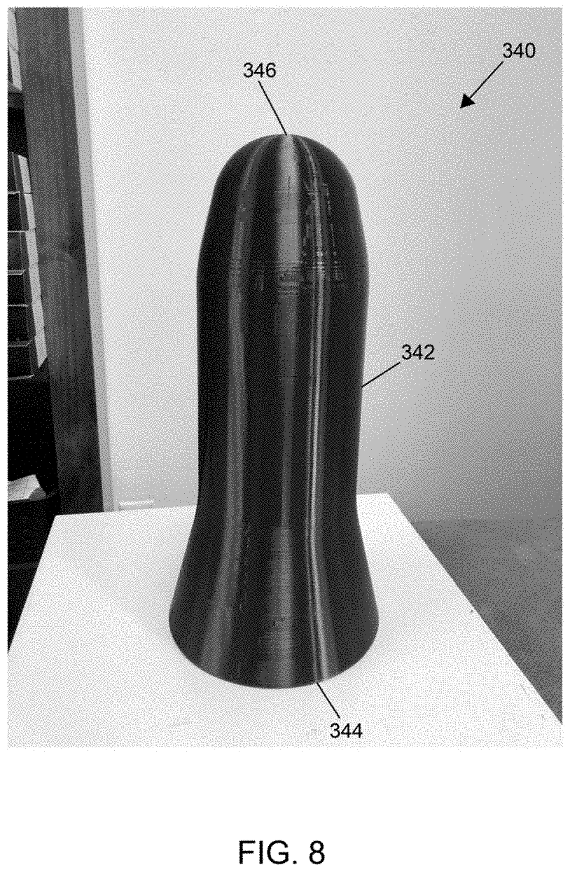

FIGS. 8 through 16 represent aspects of nonlimiting loading devices configured to be received between and below the multiple arms 16, 116, and 216 of the devices 10, 110, and 210 and to temporarily expand the arms 16, 116, and 216 such that they deflect outwards away from one another. To allow for this movement of the arms 16, 116, and 216, the arms 16, 116, and 216 may be flexible or semi-rigid, or the arms 16, 116, and 216 may be rigid with their corresponding base 16, 116, or 216 being flexible or semi-rigid.

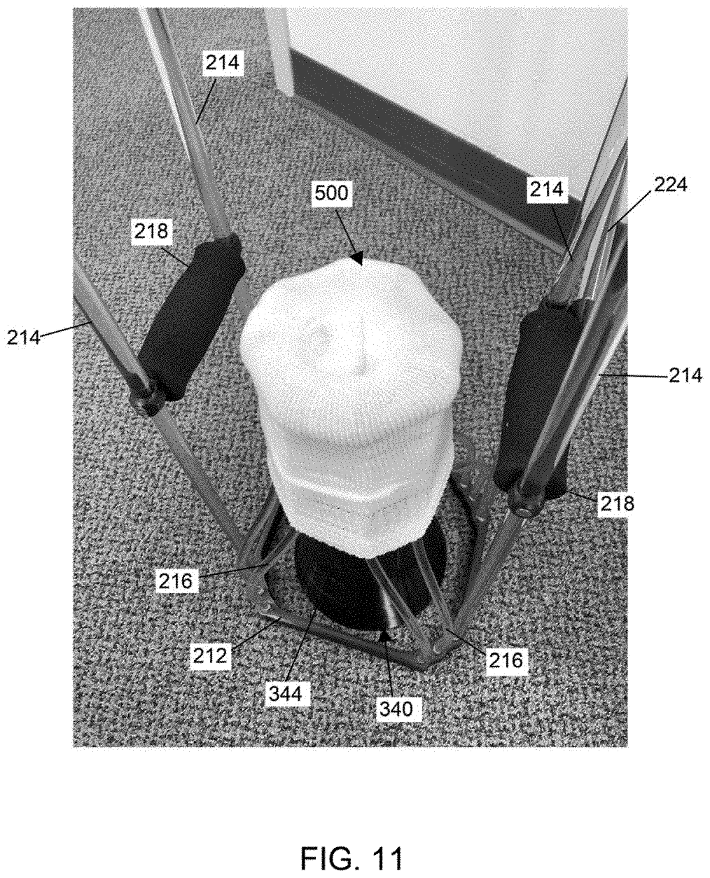

FIGS. 8 and 11 represent a first nonlimiting embodiment of a loading device 340 that includes a body comprising a cylindrical midsection 342, a conical base 344, and a rounded or cylindrical paraboloid-shaped top 346. The relatively wide base 344 promotes a stable upright resting position during use. The top 346 is rounded and/or tapered for ease of entry into the underside of the device 10, 110, or 210 between the arms 16, 116, or 216. For convenience, use of the loading device 340 will be described in reference to the device 210 of FIG. 5. However, it should be understood that the general use of the loading device 340 will be identical or substantially similar if used with the other devices 10 and 110.

During use, the device 210 may be placed such that the base 212 rests on a surface, such as the ground, floor, or a table. A compression sock 500 may be located over the rollers 220 of the arms 216 such that the sock 500 is inverted, inside out, and receives, extends over, and partially encloses upper ends of the arms 216 as represented in FIGS. 10 and 11, and as described previously in reference to the device 10.

Once the inverted sock 500 has been slipped over the top of the arms 216 and retained thereon, the loading device 340 may be located in an upright position on the surface and the entire device 210 may be lifted and located over the loading device 340 such that the loading device 340 is received within an opening in the base 212. The user may press downward on the device 210 such that the loading device 340 is forced between the arms 216 thereby spreading the arms 216 laterally and widening the opening for entry of the user's toe into the sock 500. Use of the loading device 340 is optional but prefer as it may be used to counter the compressive forces of the sock 500 with reduced effort by the user. FIG. 11 represents the loading device 340 positioned between the arms 216.

After the sock 500 has been located on the device 210 as described above, the user may insert their foot, toes first, into the opening in the sock 500 between the arms 216. As the user pushes their foot down into the toe of the sock 500, between the arms 216, and along the longitudinal axis 230, each of the arms 216 flexes radially outward from the longitudinal axis 230 allowing the foot and then leg to pass through the opening adjacent the rollers 220 and toward the opening of the base 212.

As the user inserts their foot into the device 210, the sock 500 will slide off the arms 216 and simultaneously cover their foot. The user may continue to insert their foot into the device 210 until the inverted, gathered sock 500 entirely slips over the rollers 220 of the arms 216 and onto their foot and leg. Once the sock 500 is entirely located on the user's foot, the user may retract their leg out of the device 210 by raising their leg and/or pushing down or away with the handles 218.

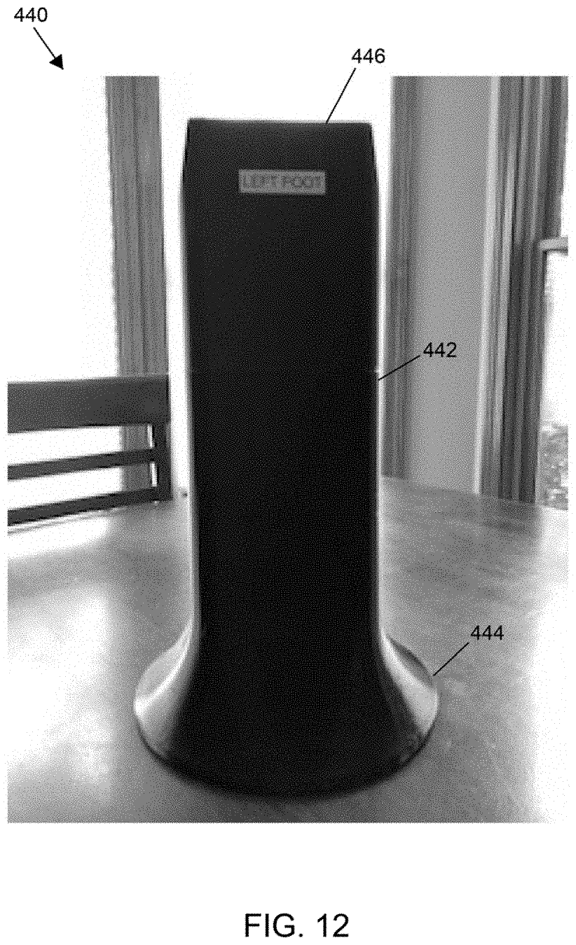

FIGS. 12 through 16 represent an alternative loading device 440 that may be used with the devices 10, 110, and 210 in substantially the same manner as described above with reference to the loading device 340 and the device 210. The loading device 440 includes a body comprising a rectangular cuboid midsection 442, a conical base 444, and an inwardly tapered top 446. Similar to the previously described loading device 340, the relatively wide base 444 of the loading device 440 promotes a stable upright resting position during use, and the top 446 is tapered for ease of entry into the underside of the device 10, 110, or 210 between the arms 16, 116, or 216.

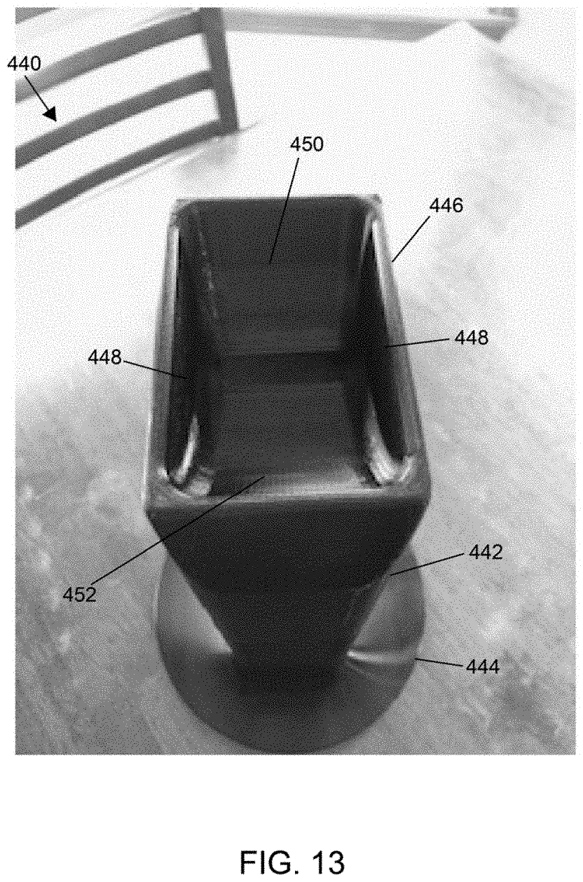

Unlike the loading device 340 which can be used from any angle due to its rounded top 346, the loading device 440 requires specific positions in which it must be located depending on whether the user is applying the sock 500 to their left or right foot. As represented in FIGS. 13 through 16, the top 446 of the loading device 440 includes a recess extending between the four sides thereof. More specifically, the recess is defined between a pair of oppositely-disposed vertical surfaces 448 and oppositely-disposed curved surfaces 450 and 452. The curved surfaces 450 and 452 are referred to herein as the medial and lateral surfaces, respectively, due to their positioning relative to the user's foot during use. Notably, the medial surface 450 has a steeper curved surface than the lateral surface 452 which has a relatively shallow curved surface.

While applying the sock 500 as described above with the device 10, 110, or 210, the loading device 450 is preferably positioned such that as the user simultaneously inserts their foot into the sock 500 and the recess of the loading device 450, the medial surface 450 is adjacent the user's big toe and the lateral surface 452 is adjacent the user's little toe. With this positioning, it is possible for the user to apply the sock 500 while apply little to no pressure on the toes of the user's foot. Optionally, the loading device 440 may include labels or markings indicating the preferred positioning of the loading device 440 based on which foot the user intends to apply the sock 500, as represented in FIGS. 12, 14, and 15.

While the invention has been described in terms of particular embodiments, it should be apparent that alternatives could be adopted by one skilled in the art. For example, the devices and their components could differ in appearance and construction from the embodiments described herein and shown in the drawings, functions of certain components of the devices could be performed by components of different construction but capable of a similar (though not necessarily equivalent) function, and various materials could be used in the fabrication of the devices and/or their components. It should also be understood that the phraseology and terminology employed above are for the purpose of describing the disclosed embodiments, and do not necessarily serve as limitations to the scope of the invention. In addition, the invention encompasses additional embodiments in which one or more features or aspects of different disclosed embodiments may be combined. Therefore, the scope of the invention is to be limited only by the following claims.

1. A device for assisting a user in covering the user's foot with a compression sock, the device comprising:
a base;
a longitudinal axis sufficiently coinciding with a center of the base so that the device is stable when the device is supported by the base on a surface;
at least two side members secured to the base on opposite sides thereof, each of the side members extending from the base along the longitudinal axis and having a handle; and
multiple arms extending from the base in a direction along the longitudinal axis and between the side members, each of the arms having a midpoint that curves inward toward the longitudinal axis of the device and a distal end, the arms in combination forming a composite shape that has a midportion that curves inward toward the longitudinal axis of the device to define a neck, defines an interior that is surrounded by the midportions of the arms and within which the longitudinal axis of the device is disposed, and defines an opening surrounded by the distal ends of the arms.

2. The device of claim 1, wherein the distal ends of the arms include a low friction exterior surface to promote sliding of the sock thereover.

3. The device of claim 1, further comprising a central opening in the base.

4. The device of claim 1, wherein each of the arms further comprise an upper portion that curves outward away from the longitudinal axis, the composite shape has an upper portion that flares outward away from the longitudinal axis, and the interior is surrounded by the upper portions of the arms.

5. The device of claim 1, further comprising spherical balls located on the distal ends of the arms.

6. The device of claim 1, further comprising soft flexible members located on the distal ends of the arms.

7. The device of claim 1, further comprising rotatable rollers located on the distal ends of the arms.

8. The device of claim 7, wherein each of the rotatable rollers is connected to two of the arms.

9. The device of claim 1, further comprising shafts pivotally coupled to the side members at a first end of the shafts and having handles on second ends of the shafts.

10. The device of claim 1, further comprising holders configured to releasably couple to one or more of the arms of the sock application device and a fastener of the holders configured to releasably couple to a sock.

11. The device of claim 1, further comprising a loading device configured to be received between and below the multiple arms of the sock application device and expand the arms such that they deflect outwards away from the longitudinal axis.

12. The device of claim 11, wherein the loading device includes a body comprising a cylindrical midsection, a conical base, and a rounded or cylindrical paraboloid-shaped top.

13. The device of claim 11, wherein the loading device includes a body comprising a rectangular cuboid midsection, a conical base, an inwardly tapered top, and a recess in the top defined between a pair of oppositely-disposed vertical surfaces and oppositely-disposed first and second curved surfaces, wherein the first curved surface is steeper than the second curved surface.

14. A method of covering at least a portion of a user's leg with a compression sock that has a foot portion including a toe portion, has a leg portion extending from the foot portion, and has an upper end that defines an opening into an interior of the compression sock, the method comprising:
providing a device comprising a base, a longitudinal axis sufficiently coinciding with a center of the base so that the device is stable when the device is supported by the base on a surface, at least two side members secured to the base on opposite sides thereof, each of the side members extending from the base along the longitudinal axis and having a handle, and multiple arms extending from the base in a direction along the longitudinal axis and between the side members, each of the arms having a midpoint that curves inward toward the longitudinal axis of the device and a distal end, the arms in combination forming a composite shape that has a midportion that curves inward toward the longitudinal axis of the device to define a neck, defines an interior that is surrounded by the midportions of the arms and within which the longitudinal axis of the device is disposed, and defines an opening surrounded by the distal ends of the arms;
placing at least a portion of the compression sock within the interior of the composite shape;
expanding the opening of the compression sock radially outward and over the distal ends of the arms;
pushing the upper end of the compression sock over the arms toward the base of the device such that at least the upper portion of the composite shape defined by the arms is surrounded by the compression sock and the foot portion of the compression sock is adjacent the opening of the composite shape; and
inserting the user's foot, toes first, into the foot portion of the compression sock at the opening of the composite shape and moving the foot through the interior of the composite shape, along the longitudinal axis of the device, and toward the base of the device;
wherein as the user inserts the user's foot through the interior of the composite shape toward the base of the device, the compression sock is applied to the user's foot and then the user's leg as the compression sock slides over the arms and the distal ends thereof until the compression sock and the upper end thereof entirely slips over the distal ends of the arms and onto the user's leg.

15. The method of claim 14, wherein as the user inserts their foot between the arms, each of the arms flex radially outward from the longitudinal axis allowing the foot to further enter the device toward the base.

16. The method of claim 14, further comprising placing the base of the device on a surface.

17. The method of claim 14, further comprising gripping the handles on the side members to further stabilize the device and/or promote insertion of the user's foot into the device.

18. The method of claim 14, further comprising removing the user's leg from the interior of the composite shape of the device by raising their leg out from between the distal ends of the arms.

19. The method of claim 14, further comprising locating a loading device between and below the multiple arms of the sock application device and expanding the arms therewith such that they deflect outwards away from the longitudinal axis after the sock has been slipped over distal ends of the arms.

20. The method of claim 19, further comprising simultaneously inserting the user's foot into the opening of the sock and a recess of the loading device such that the user's big toe is adjacent a first curved surface within the recess and the user's little toe is adjacent a second curved surface within the recess, wherein the first curved surface is steeper than the second curved surface.
